(19) United States
(12) Patent Application Publication
(10) Pub. No.: US 2011/0274725 A1
(43) Pub. Date: Nov. 10, 2011

(54) DECOMPOSABLE BIOCOMPATIBLE HYDROGELS AND SYSTEM AND METHOD FOR USING SAME

(76) Inventors: Louis Breton, Vail, AZ (US); Jeffrey Katz, Tucson, AZ (US); Mark Banister, Tuscon, AZ (US)

(21) Appl. No.: 13/119,416

(22) PCT Filed: Sep. 16, 2009
(86) PCT No.: PCT/US09/57180

§ 371 Date: Jun. 3, 2011

Related U.S. Application Data
(60) Provisional application No. 61/097,491, filed on Sep. 16, 2008.

(51) Int. Cl. A61K 9/00; A61K 38/43; A61P 27/02; A61P 29/00; A61P 35/00; A61K 31/74; A61P 31/00

(52) U.S. Cl. 424/400; 424/78.04; 424/94.1

(57) ABSTRACT

Biocompatible, triggerable degradation, polymerizable hydrogels, uses and delivery devices are disclosed. These hydrogels are at least substantially water soluble macromers, having a variety of uses especially for ocular therapy. The macromers include at least one water soluble region, at least one region which is degradable via a triggering event, usually by hydrolysis, and at least two free radical-polymerizable regions. The regions can, in some embodiments, be both water soluble and trigger able degradable. The macromers are polymerized by exposure of the polymerizable regions to free radicals generated by photosensitive chemicals and dyes. An advantage of these polymer hydrogels is that they can be polymerized rapidly in an aqueous surrounding. Precisely conforming, semi-permeable, biodegradable films or membranes can thus be formed on tissue in situ to serve as biodegradable barriers, as carriers for living cells or other biologically active materials, and as surgical adhesives for the eye.

The invention pertains to biocompatible hydrogels, and more particularly to biocompatible polymerizable hydrogels and to systems and methods for using same. The invention has particular utility in connection with biocompatible hydrogels and their use as biodegradable barriers, e.g. for treating the eyes, and will be described in connection with such utility, although other utilities are contemplated.

Existing polymer biomaterials—including ophthalmic materials—while generally useful for specific functions are subject to limitations. Existing ophthalmic biomaterials (e.g. intraocular lenses, keratoprostheses and contact lenses) exhibit a satisfactory degree of ocular biocompatibility but they must be removable for long-term use. In the case of many existing biodegradable products degradation is relatively slow over time and may be influenced by uncontrolled environmental factors. Therefore, a polymer that degrades rapidly and at will upon introduction of a triggering solution or event would be very advantageous for ocular therapies.

Fibrin gels have been used extensively in Europe as sealants and adhesives in surgery (References 1 and 2 below). However, they have not been used extensively in the United States due to concerns relating to disease transmission from blood products. Synthetic polymers have been explored as adhesives (Reference 3 below), but these materials have been associated with local inflammation, cytotoxicity, and poor biocompatibility.

Prevention of Postoperative Adhesions.

Formation of post-surgical adhesions involving organs of the peritoneal cavity and the peritoneal wall is a frequent and undesirable result of abdominal surgery. Surgical trauma to the tissue caused by handling and drying results in release of a serosanguinous (proteinaceous) exudate that tends to collect in the pelvic cavity (Reference 4 below). If the exudate is not absorbed or lysed within this period it becomes ingrown with fibroblasts, and subsequent collagen deposition leads to adhesion formation. Numerous approaches to eliminate adhesion formation have been attempted with limited success in most cases. Approaches have included lavage of the peritoneal cavity, administration of pharmacological agents, and the application of barriers to mechanically separate tissues.

Solutions of Poloxamer 407 have been used for the treatment of adhesions with some success. Poloxamer is a copolymer of ethylene oxide and propylene oxide and is soluble in water; the solutions are liquids at room temperature. References 5 and 6 below examined Poloxamer solutions in peritoneal adhesion models and observed statistically significant reductions in adhesions; however, they were unable to eliminate adhesions, perhaps because of limited adhesion and retention on the injury site.

Oxidized regenerated cellulose has been used extensively to prevent adhesions and is an approved clinical product, trade-named Interceed TCY. This barrier material has been shown to be somewhat effective in rabbits (References 7 and 8 below). It was shown to be more effective if pretreated with heparin, but was still unable to completely eliminate adhesions (Reference 9 below).

An ideal material barrier would not evoke an adhesion response itself, stay in place without suturing (Reference 10 below), degrade over a few weeks' time, effectively reduce adhesions to very low extent, and be capable of delivering a drug to the local site of application for several days' time. None of the approaches developed and described to date meet these requirements.

The field of biodegradable polymers has developed rapidly since Kulkarni et al. first reported the synthesis and biodegradability of polylactic acid in reference 11 below. Several other polymers are known to biodegrade, including polyanhydrides and polyorthoesters, which take advantage of labile backbone linkages, as reported by Domb et al., and Heller et al. in references 12 and 13 below. Since it is desirable to have polymers that degrade into naturally occurring materials, polyaminoacids have been synthesized, as reported by Miyake et al. in 1974 for in vivo use. This was the basis for using polyesters (Reference 14 below) of α-hydroxy acids (viz., lactic acid, glycolic acid), which remain the most widely used biodegradable materials for applications ranging from closure devices (sutures and staples) to drug delivery systems (References 15 and 16 below).

The time required for a polymer to degrade can be tailored by selecting appropriate monomers. Differences in crystallinity also alter degradation rates. Due to the relatively hydrophobic nature of these polymers, actual mass loss only begins when the oligomeric fragments are small enough to be water-soluble. Hence, initial polymer molecular weight influences the degradation rate and limits application. A method to trigger rapid degradation at a chosen time is needed and more desirable.

Degradable polymers containing water-soluble polymer elements have been described. Sawhney et al., copolymerized lactide, glycolide and α-caprolactone with PEG to increase its hydrophilicity and degradation rate. (Reference 17 below). Casey et al. synthesized a PGA-PEG-PGA block copolymer, with PEG content ranging from 5-25% by mass. (Reference 18 below) Casey et al. also reports synthesis of PGA-PEG diblock copolymers, again with PEG ranging from 5-25%. Churchill et al. described non-crosslinked materials with MW in excess of 5,000, based on similar compositions with PEG; although these materials are not water soluble. (Reference 19 below). Reference 20 above described PLA-PEG copolymers that swell in water up to 60%; these polymers also are not soluble in water, and are not crosslinked. The features that are common to these materials is that they use both water-soluble polymers and degradable polymers, and that they are insoluble in water, collectively swelling up to about 60%.

Degradable materials of biological origin are well known, for example, crosslinked gelatin. Hyaluronic acid has been crosslinked and used as a degradable swelling polymer for biomedical applications (Reference 21-23 below).

Most hydrophilic drugs are mechanically dispersed as suspensions within solutions of biodegradable polymers in organic solvents. Protein and enzyme molecular conformations are frequently different under these circumstances than they would be in aqueous media. An enzyme dispersed in such a hydrophobic matrix is usually present in an inactive conformation until it is released into the surrounding aqueous environment subsequent to polymer degradation.

Polymer synthesis, triggerable degradation and local synthesis biodegrading polymers currently suggested for short-term macromolecular drug release may raise local concentrations of potentially hazardous acidic degradation byproducts. Further, all biocompatible degradable synthetic polymers reported thus far can only be processed inorganic solvents and all biodegradable polymers are synthesized under conditions that are not amenable to polymerization in vivo. Thus, it has not been possible to make implantable materials as precisely conformed barriers, shaped articles, or membranes capable of delivering bioactive materials to the local tissue.

In summary, several lavage/drug/material approaches have been explored, but none of these approaches has been able to eliminate substantially completely adhesions.

So as to reduce the complexity and length of the Detailed Specification, and to fully establish the state of the art in certain areas of technology, Applicant(s) herein expressly incorporate(s) by reference all of the following materials identified in each numbered paragraph below.

1. Thompson et al., 1988, “Fibrin Glue: A review of its preparation, efficacy, and adverse effects as a topical hemostat,” Drug Intell. and Clin. Pharm., 22:946.

2. Gibble et al., 1990, (1990), “Fibrin glue: the perfect operative sealant?” Transfusion, 30(8):741.

3. Lipatova, 1986, “Medical polymer adhesives,” Advances in Polymer Science 79:65-93.

4. Holtz, G., 1984.

5. Steinleitner et al. (1991) “Poloxamer 407 as an Intraperitoneal Barrier Material for the Prevention of Post surgical Adhesion Formation and Reformation in Rodent Models for Reproductive Surgery,” Obstetrics and Gynecology, 77(1):48.

6. Leach et al. (1990) “Reduction of postoperative adhesions in the rat uterine horn model with poloxamer” 407, Am. J. Obstet. Gynecol., 162(5):1317.

7. Linsky et al., 1987 “Adhesion reduction in a rabbit uterine horn model using TC-7,” J. Reprod. Med., 32:17.

8. Diamond et al., 1987 “Pathogenesis of adhesions formation/reformation: applications to reproductive surgery,” Microsurgery, 8:103) and in humans (Interceed (TC7) Adhesion Barrier Study Group, 1989.

9. Diamond et al., 1991 “Synergistic effects of INTERCEED(TC7) and heparin in reducing adhesion formation in the rabbit uterine horn model,” Fertility and Sterility, 55(2):389).

10. Holtz et al., 1982 “Adhesion induction by suture of varying tissue reactivity and caliber,” Int. J. Pert., 27:134.

11. Kulkarni et al., 1966 “Polylactic acid for surgical implants,” Arch. Surf., 93:839.

12. Domb et al., 1989 Macromolecules, 22:3200;

13. Heller et al., 1990 Biodegradable Polymers as Drug Delivery Systems, Chasin, M. and Langer, R., Eds., Dekker, N.Y., 121-161.

14. Holland et al., 1986 Controlled Release, 4:155-180.

15. U.S. Pat. No. 4,741,337 to Smith et al.;

16. Spilizewski et al., 1985 “The effect of hydrocortisone loaded poly(dl-lactide) films on the inflammatory response,” J. Control. Rel. 2:197-203.

17. Sawhney et al., (1990) “Rapidly degraded terpolymers of dl-lactide, glycolide, and ε-caprolactone with increased hydrophilicity by copolymerization with polyethers,” J. Biomed. Mater. Res. 24:1397-1411.

18. U.S. Pat. No. 4,716,203 to Casey et al. (1987)

19. U.S. Pat. No. 4,526,938 to Churchill et al. (1985)

20. Cohn et al. (1988) J. Biomed. Mater. Res. 22:993-1009.

21. U.S. Pat. No. 4,987,744 to della Valle et al.,

22. U.S. Pat. 4,957,744 to Della Valle et al.

23. “Surface modification of polymeric biomaterials for reduced thrombogenicity,” Polym. Mater. Sci. Eng., 62:731-735.

The present invention provides, among other things, biocompatible degradable hydrogels of polymerized and cross-linked biocompatible materials such as epoxides, monomers, macromers, and dendrimers. The materials may comprise, for example, hydrophilic linkages, chains, monomers or oligomers capable of polymerization and cross linking, epoxies having degradable links, or monomeric or oligomeric extensions terminated on free ends with end cap reactive sites. The hydrogel typically has a hydrophilic core that may be degradable, thus combining the core and extension degrading functions of the material. These materials are typically polymerized using free radical initiators under the influence of long wavelength ultraviolet light, visible light excitation or thermal energy. They may also be polymerized via introduction of a solvent reactant or epoxy reactant and would be known to anyone familiar in the art.

The biocompatible degradable hydrogels can be carriers for biologically active materials such as hormones, enzymes, antibiotics, antineoplastic agents, and cell suspensions. Temporary preservation of functional properties of a carried species, as well as controlled release of the species into local tissues or systemic circulation is possible. Proper choice of hydrogel macromers can produce membranes with a range of permeability, pore sizes, and degradation rates suitable for a variety of applications in surgery, medical diagnosis and treatment.

Cleavable sites are incorporated into the hydrogels polymer chains or linkages. The cleavable sites are specifically incorporated to break up or degrade the polymer hydrogel rapidly and at will upon introduction of a cleavage triggering agent or event. The cleavable sites react to a degradation event initiated by the addition of an aqueous solvent or solution containing the chemical or material needed to initiate cleavage at the cleavable site within the polymer network. In a preferred embodiment as discussed below the hydrogels may also be used as a temporary protecting or therapeutic eye covering or as a pharmaceutical drug carrier that will release the drug over time or upon the degradation event. The polymer hydrogel may also be used to enhance vision temporarily for short periods of time.

Useful photo-initiators are those that can use free radical generation to initiate polymerization of the macromers without cytotoxicity and within a short time frame. Preferred initiator dyes for LWUV or visible light initiation include ethyl eosin, 2,2-dimethoxy-2-phenyl acetophenone, other acetophenone derivatives, and camphorquinone. In all cases, cross-linking and polymerization are initiated among macromers by a light-activated free-radical polymerization initiator such as 2,2-dimethoxy-2-phenylacetophenone, a combination of ethyl eosin (10⁻⁴ to 10⁻² M) and triethanol amine (0.001 to 0.1 M), xanthine dyes, acridine dyes, thiazine dyes, phenazine dyes, camphorquinone dyes, and acetophenone dyes, eosin dye with triethanolamine, 2,2-dimethyl-2-phenyl acetophenone, and 2-methoxy-2-phenyl acetophenone. Cross-linking or polymerizations can be initiated in situ by light typically having a wavelength of 320 nm or longer.

The choice of the photo-initiator is largely dependent on the photopolymerizable regions. For example, when the macromer includes at least one carbon-carbon double bond, light absorption by the dye causes the dye to assume a triplet state, the triplet state subsequently reacting with the amine to form a free radical that initiates polymerization. Preferred dyes for use with these materials include eosin dye and initiators such as 2,2-dimethyl-2-phenylacetophenone, 2-methoxy-2-phenylacetophenone, and camphorquinone. Using such initiators, copolymers may be polymerized in situ by long wavelength ultraviolet light or by laser light of about 514 nm, for example.

Initiation of polymerization may be accomplished by irradiation with light at a wavelength of between about 200-700 nm, most preferably in the long wavelength ultraviolet range or visible range, 320 nm or higher, most preferably about 514 nm or 365 rim.

There also are several photo oxidizable and photo reducible dyes that may be used to initiate polymerization, including acridine dyes such as acriblarine; thiazine dyes such as thionine; xanthine dyes such as rose bengal; and phenazine dyes, such as, methylene blue. These dyes typically are used with cocatalysts such as amines, for example, triethanolamine; sulphur compounds such as, RSO₂R¹; heterocycles such as imidazole; enolates; organometallics; and other compounds such as N-phenyl glycine also may be used. Other initiators include camphorquinones and acetophenone derivatives all of which are well known in the art.

Thermal polymerization initiator systems also may be used. Such systems that are unstable at 37° C. and would initiate free radical polymerization at physiological temperatures include potassium persulfate, with or without tetraamethyl ethylenediamine; benzoylperoxide, with or without triethanolamine; and ammonium persulfate with sodium bisulfite.

The degradation event is triggered by an introduced change in the environment of the gel, such as the addition of liquid drops of a solution that is biocompatible yet different from the existing environment. The reactive sites in the polymer network react to the introduced solution in a way that cleaves or breaks the polymer network. This is accomplished by solutions of a differing pH, salt, weak acid or oxidizing compound or other mechanism known in the art. The change in solution in and around the polymer network results in reversal of the polymer linking, cleaving of the polymer links or overstressing at the linkages or chains within the polymer creating polymer fragments which are non-toxic and easily removed from the body by passing through the tear ducts.

It is therefore an object of the present invention to provide hydrogels which are biocompatible, offer selectively triggerable degradation, and can rapidly be formed by polymerization as the product is applied, can be stored and applied already polymerized completely or stored partially polymerized until applied

A specific and preferred object of the present invention to provide a macromer solution which can be administered to the eye during surgery or outpatient procedures and polymerized as a tissue adhesive, tissue encapsulating medium, tissue support, or drug delivery medium that can be removed via triggerable degradation at will.

Yet another specific and preferred object of the present invention to provide a macromer solution for the eye which can be polymerized in a very short time frame and in very thin, or ultra thin, layers and produce clear eyesight enhancing properties alone or with the addition of light refracting materials such as but not limited to titanium oxide.

The above and other objects in one aspect may be achieved using devices involving a biocompatible hydrogel comprising a polymer wherein the polymer comprises a hydrophilic core, a polymerized material linked to the hydrophilic core made up of a series of reactive sites configured to react to a specific degradation trigger so that the polymerized material is degraded upon application of the degradation trigger.

In one embodiment the material comprises a biocompatible hydrogel having a hydrophilic core that is selectively degradable upon application of a degradation trigger.

Preferably the degradation trigger acts on a degradable biocompatible hydrogel made up of a solution of differing pH, a salt, a salt solution, a weak acid solution, or an oxidizing compound.

If desired the degradable biocompatible hydrogel further comprises a pharmaceutical configured to be released upon degradation of the polymerized material.

Preferably the polymerized material is a material that is degraded upon application of the degradation trigger such that the degraded material is absorbed through the tear ducts of the eye.

In another preferred embodiment the macromer solution is administered to the eye and comprises a pre-polymer material that is polymerized upon application of a specific polymerization trigger wherein the pre-polymer material is designed to be degradable after polymerization upon application of a degradation trigger.

If desired, the macromer solution produces eyesight-enhancing properties upon polymerization by a polymerization trigger.

Also, if desired, the macromer solution may be polymerized by application of a polymerization trigger where the pre-polymer material is made up of a photo-initiator that begins polymerization of the pre-polymer material upon exposure to light, heat, or a biocompatible reagent.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the use of the words “function,” “means” or “step” in the Detailed Description or Description of the Drawings or claims is not intended to somehow indicate a desire to invoke the special provisions of 35 U.S.C. §112, ¶ 6, to define the invention. To the contrary, if the provisions of 35 U.S.C. §112, ¶ 6 are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. §112, ¶ 6. Moreover, even if the provisions of 35 U.S.C. §112, ¶ 6 are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

Non-limiting examples of biocompatible and functional monomers and polymers useful in the practice of the present invention include N-vinyl amides, such as N-methyl vinyl acetamide. These structures provide useful complements to the structurally isomeric substituted acrylamides, such as N,N-dimethyl acrylamide. Similarly, N-acryloyl morpholine and N-hydroxyethyl acrylamide provide examples of less widely used members of the acrylamide family that, in turn, usefully supplement the range of hydrogel polymer precursors. Other examples are ionic and zwitterionic monomers, 2-Acrylamido-2-methylpropane sulphonic acid (AMPS) and its sodium salt NaAMPS. Other sulphonate monomers provide valuable structural complements. These include the sulphopropyle acrylates, itaconates and methacrylates, SPA SPI and SPM. Zwitterionic monomer, N,N-dimethyl-N-(2-acryloylethyl)-N-(3-sulfopropyl) ammonium betaine (SPDA) [Raschig, GMBH]. This monomer can be structurally compared to the methacroyl derivative of phosphitadyl choline (MPC). SPDA and MPC are both acrylate-based monomers that contain quaternary nitrogen groups. The major difference is that MPC has a phosphorylcholine group while SPDA has a sulphonate group and these are positioned differently in the molecule. Taken together these monomers provide versatile building blocks for biocompatible polymer materials.

For descriptive purposes, as used herein a macromer is essentially an assembly of pre-polymerized monomers that has been modified to enable it to act as a monomer. Macromers advantageously overcome the problems of toxicity encountered with low molecular weight monomers and have much lower polymerization isotherms—valuable characteristics in injectable tissue repair systems. Multi-functional macromers—chains that contain several polymerizable double bonds—make effective hydrogel cross-linking agents. More importantly, purpose-designed macromers can be used as interpenetrates. In this way the properties of hydrogels can be enhanced through the introduction of networks of varying degrees of strength within the hydrogel network. This is typically done to incorporate a property that the polymer network is desired to have such as hydrophobic or hydrophilic properties, biodegradability etc. and are well known in the art.

Aliphatic-aromatic co-polyesters combine the excellent material properties of aromatic polyesters (e.g. PET) and the biodegradability of aliphatic polyesters. They are soft, pliable and have good tactile properties.

Aliphatic polyesters are biodegradable but often are lacking in good thermal and mechanical properties. While, vice versa, aromatic polyesters (like PET) have excellent material properties, but are resistant to microbial attack. Typical aliphatic polyesters include polyhydroxy butyrate, polycaprolactone, polylactic acid and polybutylene succinate. Aliphatic polyesters degrade like starch or cellulose to produce non-humic substances such as CO₂ and methane.

Copolyesters combine aromatic esters with aliphatic esters or other polymer units (e.g. ethers and amides) and thereby provide the opportunity to adjust and control. Polyethylene tetraphalate (PET) is a rigid polymer to which aliphatic monomers such as PBAT (polybutylene adipate/terephthalate) and PTMAT (polytetramethylene adipate/terephthalate)can be added to enhance biodegradability.

Up to three aliphatic monomers can be incorporated into the PET structure to create weak spots in the polymeric chains that make them susceptible to degradation through hydrolysis. This degradation can be further enhanced by the addition of salts, acids or alkaline attraction sites such as amine or hydroxyl groups at linkages within the polymer network or chain. The electrostatic repulsion or attraction can itself be the linkage cleaving mechanism.

Polybutylene succinate (PBS) and polybutylene succinate adipate (PBSA) typify biodegradable synthetic aliphatic polyesters. Adipate co-polymers typically are added to the PBS polymer to make its use more economical.

Polycaprolactone is a biodegradable thermoplastic polymer derived from the chemical synthesis of crude oil. Although not produced from renewable raw materials, it is fully biodegradable.

Polyesters are polymers with ester groups in their backbone chains. Polyesters will degrade eventually, with hydrolysis being the dominant mechanism. Polyhydroxyalkanoates (PHA) are linear aliphatic polyesters produced in nature by bacterial fermentation of sugar or lipids. More than 100 different monomers can be combined within this family to give materials with extremely different properties. They can be either thermoplastic or elastomeric materials, with melting-points ranging from 40 to 180° C. The most common type of PHA is PHB (polybeta-hydroxybutyrate). PHB has properties similar to those of polypropylene, but is stiffer and more brittle. Polyhydroxybutyrate-valerate copolymer (PHBV) is a PHB copolymer which is less stiff and tougher, and is typically used as packaging material.

Polylactic acid (PLA) is another biodegradable polymer that is derived from lactic acid. PLA resembles clear polystyrene and provides good aesthetics (gloss and clarity), but is stiff and brittle and needs modification for most practical applications (e.g. plasticizers increase its flexibility).

Biodegradable polylactic acid aliphatic copolymer (CPLA) is a mixture of polylactic acid and other aliphatic polyesters. It can be either a hard plastic (similar to PS) or a soft flexible one (similar to PP) depending on the amount of aliphatic polyester present in the mixture.

Polyvinyl alcohol (PVOH) is a synthetic, water-soluble and readily biodegradable polymer.

Starch composites can be used as a biodegradable additive or replacement material in traditional oil-based commodity plastics. If starch is added to petroleum derived polymers (e.g. PE), it facilitates disintegration of the blend, but not necessarily biodegradation of the polyethylene component. Starch accelerates the disintegration or fragmentation of the synthetic polymer structure. Microbial action consumes the starch, thereby creating pores in the material which weaken it and enable it to break apart.

Also called plasticized starch materials, such composites exhibit mechanical properties similar to conventional plastics such as PP, and are generally resistant to oils and alcohols though they degrade when exposed to hot water. Their basic content (40-80%) is corn starch, a renewable natural material. The balance is performance-enhancing additives and other biodegradable materials added to the polymer chains.

Starch composites of (90% Starch) are usually referred to as thermoplastic starch. They are stable in oils and fats. However, depending on the type, they can vary from stable to unstable in hot/cold water. They can be processed by traditional techniques for plastics.

In one embodiment of the invention, the biocompatible hydrogel has solubility in an aqueous solution of predetermined pH comprising at least one water soluble region or active groups, at least one triggered degradation region which is hydrolysable such as a starch or polyester or polyvinyl alcohol, preferably under invivo conditions, and free radical polymerizing end groups having the capacity to form additional covalent bonds resulting in macromer interlinking, where the polymerizing end groups are separated from each other by at least one triggerable degradation region.

By applying aqueous solution or solvent of differing properties (such as pH or a salt), to the polymer after it has been applied to the eye, a triggering event is created at the time that rapid degradation is desired. The polymers triggerable degradation region would react to this event by cleaving or reversal of the linking allowing rapid degradation of the polymer. The degradation need not be complete—the polymer only needs break apart to a size that enables the ability of the degraded polymer to pass through the tear ducts of the eye so it can in turn pass through the digestive tract of the body. The biocompatible, triggered-degradation hydrogel may also be used as a drug carrying therapy that is applied to the eye so it releases the drug over time or holds the drug in place on the eye until the degradation event. There are numerous examples of therapeutic drugs that may be combined with hydrogels whereby the degradation event may be designed to release a drug or therapeutic agent to the eye only at the time of the triggering event.

Another embodiment of the invention includes a polymer hydrogel where the water soluble region is attached to a triggered degradation region, at least one polymerizing end group attached to the water soluble region, and at least one polymerizing end group attached to the triggerable degradation region.

Yet another embodiment is a polymer hydrogel where the water-soluble region forms a central core, at least two triggerable degradable regions attached to the core, and the polymerized end groups attached to the trigger able degradable regions.

Yet another embodiment is a polymer hydrogel where the triggerable degradable region is a central core, at least two water soluble regions are attached to the core, and a polymerizing end group is attached to each water soluble region.

Yet another embodiment is a polymer hydrogel where the water soluble region is a macromer backbone, the triggerable degradable region is a branch or graft attached to the macromer backbone, and polymerizing end groups are attached to the triggering degradable regions.

Yet another embodiment is a polymer hydrogel where the triggerable degradable region is a macromer backbone, the water soluble region is a branch or graft that is attached to the degradable backbone, and polymerizable end groups are attached to the water soluble branches or grafts.

Yet another embodiment is a polymer hydrogel where the water soluble region is a star backbone, the triggerable degradable region is a branch or graft attached to the water soluble star backbone, and at least two polymerizable end groups are attached to a degradable branch or graft.

Yet another embodiment is a polymer hydrogel where the triggerable degradable region is a star or highly branched backbone, the water soluble region is a branch or graft attached to the degradable star backbone, and two or more polymerizable end groups are attached to the water soluble branch or graft.

Yet another embodiment is a polymer hydrogel where the water soluble region is also the triggerable degradable region where the water soluble region is over stressed and cleaves from addition of water

Yet another embodiment is a polymer hydrogel where the water-soluble region is also the triggerable degradable region, one or more additional degradable regions are grafts or branches upon the water-soluble region.

Yet another embodiment is a polymer hydrogel comprising a water soluble core region, at least two triggerable degradable extensions on the core, and an end cap on at least two of the triggerable degradable extensions, wherein the core comprises poly(ethylene glycol); each extension comprises biodegradable poly(α-hydroxy acid); and each end cap comprises an acrylate oligomer or monomer. In a preferred embodiment, the poly(ethylene glycol) has a molecular weight between about 400 and 30,000 Da; the poly(hydroxy acid) oligomers have a molecular weight between about 200 and 1200 Da; and the acrylate oligomer or monomer have a molecular weight between about 50 and 200 Da.

Yet another embodiment is a polymer hydrogel where each extension comprises biodegradable poly(hydroxy acid); and each end cap comprises an acrylate oligomer or monomer and the addition of a alkaline aqueous solution triggers degradation

Yet another embodiment is a polymer hydrogel where the polymerizable end groups contain a carbon-carbon double bond capable of cross-linking and polymerizing macromers.

Yet another embodiment is a polymer hydrogel where crosslinking and polymerization of the macromer can be initiated by a light-sensitive free-radical polymerization initiator with or without a cocatalyst, further comprising a free radical polymerization initiator.

Yet another embodiment is a polymer hydrogel where the triggerable degradable region is selected from the group consisting of poly (hydroxy acids), poly(lactones), poly(amino acids), poly(anhydrides), poly(orthoesters), poly(phosphazines), and poly(phosphoesters), poly(ε-caprolactone), poly (δ-valerolactone) or poly(λ-butyrolactone).

Yet another embodiment is a polymer hydrogel where the trigger able degradable region is a poly(α-hydroxy acid) selected from the group consisting of poly(glycolic acid), poly(DL-lactic acid) and poly(L-lactic acid).

Yet another embodiment is a polymer hydrogel where the water soluble region is selected from the group consisting of poly(ethylene glycol), poly(ethylene oxide), poly(ether amines), poly(vinyl alcohol), poly(vinylpyrrolidone), poly(ethyloxazoline), poly(ethylene oxide)-copoly(propyleneoxide) block copolymers, polysaccharides, carbohydrates, proteins, and combinations thereof.

Yet another embodiment is a polymer hydrogel of any of the other embodiments, further comprising biologically active molecules selected from the group consisting of proteins, carbohydrates, nucleic acids, organic molecules, inorganic biologically active molecules, cells, tissues, and tissue aggregates.

In preferred embodiments, the core water soluble region can consist of poly(ethylene glycol), poly(ethylene oxide), poly(vinyl alcohol), poly(vinylpyrrolidone), poly(ethyloxazoline), poly(ethylene oxide)-co-poly(propyleneoxide) block copolymers, polysaccharides or carbohydrates such as hyaluronic acid, dextran, heparan sulfate, chondroitin sulfate, heparin, or alginate, proteins such as gelatin, collagen, albumin, ovalbumin, or polyamino acids.

The triggerable degradable region is preferably hydrolyzable under in vivo conditions. For example, the hydrolyzable group or groups may be polymers and oligomers of glycolide, lactide, ε-caprolactone, other hydroxy acids, and other biologically degradable polymers that yield materials that are non-toxic or present as normal metabolites in the body. Preferred poly(α-hydroxy acid)s are poly(glycolic acid), poly(DL-lactic acid) and poly(L-lactic acid). Other useful materials include poly(amino acids), poly(anhydrides), poly(orthoesters), poly(phosphazines) and poly(phosphoesters). Polylactones such as poly(ε-caprolactone), poly(ε-caprolactone), poly(δ-valerolactone) and poly(gamma-butyrolactone), for example, are also useful. The triggerable degradable regions may have a degree of polymerization ranging from one up to values that would yield a product that was not substantially water soluble. Thus, monomeric, dimeric, trimeric, oligomeric, and polymeric regions may be used.

Triggerable degradable regions can be constructed from polymers or monomers using linkages susceptible to biodegradation, such as ester, peptide, anhydride, orthoester, phosphazine and phosphoester bonds. The polymerizable regions are preferably polymerizable by photoinitiation by free radical generation, most preferably in the visible or long wavelength ultraviolet radiation. The preferred polymerizable regions are acrylates, diacrylates, oligoacrylates, methacrylates, dimethacrylates, oligomethoacrylates, or other biologically acceptable photopolymerizable groups.

The cleaning or degradation of the polymer may also be initiated by irradiation of light such as of UV or IR spectrum, or a combination of solution and irradiation applied together. Other initiation chemistries may be used besides photoinitiation. These include, for example, water and amine initiation schemes with epoxides, an example of such is a gel which was made with branched Polyethylenimine, reacted with water, a cleavable heterobifunctional crosslinker such as N-(3-Dimethylaminopropyl)-N′-ethylcarbodiimide hydrochloride and an epoxide such as polyethylene glycol diglycidyl ether. The result is a clear, rapidly polymerizing gel that forms a thin film on the eye and when additional fluid is added it degrades rapidly. Another useful cleaveable crosslinker is Bromoacetic acid N-hydroxysuccinimide ester, a heterobifunctional cross-linking reagent which allows bromoacetylation of primary amine groups. Ethylene glycol-bis(succinic acid N-hydroxysuccinimide ester) is another crosslinker with cleavable sites. These are just a few examples of biocompatable crosslinkers that can be utilized and are not meant to limit the scope of the invention in any way. Additionally isocyanate and isothiocyanate containing macromers may be used as the polymerizable regions. Triggerable degradable regions also may be constructed using molecular engineered methods such as but not limited to dendritic synthesis and click chemistry.

Additional Uses of the Triggered Degradation Hydrogels

For lens applications, the monomer mixtures employed in the invention include a monomeric material of this invention mixed with various conventional lens-forming monomers. The lens-forming monomers preferably are monomers that are polymerizable by free radical polymerization, generally including an activated unsaturated radical, and most preferably an ethylenically unsaturated radical. As used herein, the term“monomer”and like terms denote relatively low molecular weight compounds that are polymerizable by free radical polymerization, as well as higher molecular weight compounds also referred to as“prepolymers”,“macromonomers”, and similar terms. Optionally, the initial monomeric mixture may also include additional materials such as solvents, colorants, toughening agents, UV-absorbing agents and other materials such as those known in the contact lens art.

Delivery System

Hydrogels can be gelled as applied from a delivery device using a single or separate compartments housing water-soluble precursors. The polymer solution is squeezed or pumped through a flexible hollow channel applicator tip that allows mixing of the solution in the case of separated solutions prior to polymerization. The delivery device at the correct position houses an ultra-violet or other wavelength light source such as a light emitting diode powered via a battery. The light source illuminates and reacts the initiators to thereby begin polymerization of the solution, and the resulting formation of hydrogel takes place as the hydrogel is applied. The light source is positioned in the applicator so no harmful UV light is applied directly to the eye.

Another delivery embodiment is a container that when squeezed or activated mixes the unreacted solutions or monomers to start the polymerization reaction via methods such as free radical initiation or catalytic initiation that occurs prior to or as the material is applied to the eye.

Yet another delivery embodiment is a container that houses the triggerable degradable polymer hydrogel already polymerized, such as a thermally responsive gel. In this embodiment the polymerized gel changes to the needed viscosity as it is applied. In other embodiments the triggerable degradable hydrogel would not change viscosity and be already polymerized, yet viscous enough to be applied as is and only thickens or sets from exposure to air or temperature or other external stimulus.

Yet another delivery embodiment is a container that has separate storage areas for both the Trigger able hydrogel and the solution to trigger the degradation.

Yet another delivery embodiment is an eye drop applicator that contains solution to be applied to eyes and an applicator tip or tip with a cover that sterilizes the applicator tip by exposing the tip to UV light. The tip cover or container contains the power source and LED to produce the light that sterilizes the applicator tip. This would be of benefit to help reduce easily transmitted eye infections or conditions such as pink eye.

While the invention has been described for use primarily as tissue glue, the invention also can be used to form a protective “lens”, e.g. to protect a surgical area or wound. Thus, the hydrogel may be applied over an abrasion, or preformed, e.g. like a contact lens. Preforming the hydrogel as a contact lens permits a patient to maintain essentially clear vision, while the hydrogel contact lens provides a sealant to protect the eye. If desired, a therapeutic agent may be incorporated into the hydrogel and released as the hydrogel is degraded. Thus, by degradation of the hydrogel, the therapeutic agents may be released over time.

A feature and advantage of the present invention is that hydrogel material of the present invention is essentially optically clear. Thus, it can be used as a shield, e.g. to protect the eye/cornea following any injury, such as following a corneal abrasion. The hydrogel material of the present invention also may be used as treatment for dry eyes so as to eliminate the necessity of constant tear drop installation to the eye. Additionally, the hydrogel material of the present invention may be used to deliver antibiotics/drugs to the corneal surface as the hydrogel can act as a sponge, imbibe antibiotic or anti inflammatory compounds, and then release slowly over time as it persists on the corneal surface. The material also advantageously may be used as a protective sealant following ocular surgeries. For example, ocular surgeries could be covered with this substance to act to prevent wound leakage and/or reduce complications of a leaky wound. The material also could be used for wound healing and promote epithelialization post ocular surgeries or injury.

Still other possibilities are possible. For example, the material could be used for treating Presbyopia by increasing the index of refraction of the hydrogel so that after installation of a drop, a person would be able to read at near for a limited time without the use of reading glasses.

We claim:

1. A biocompatible hydrogel comprising a polymer which comprises:
a hydrophilic core;
a polymerized and cross-linked material chemically linked to the hydrophilic core wherein the polymerized and cross-linked material comprises reactive sites configured to react to a specific degradation trigger and wherein the polymerized and cross-linked material may be selectively degraded upon application of a biocompatible degradation trigger.

2. The biocompatible hydrogel of claim 1, wherein the degradation trigger comprises a aqueous-based material.

3. The biocompatible hydrogel of claim 1, wherein the hydrophilic core is also selectively degradable upon application of a degradation trigger.

4. The biocompatible hydrogel of claim 1, wherein the hydrophilic core is also selectively degradable upon application of a second degradation trigger.

5. The biocompatible hydrogel of claim 1, wherein the degradation trigger is selected from the group consisting of a solution of differing pH, a salt, a salt solution, a weak acid solution, and an oxidizing compound.

6. The biocompatible hydrogel of claim 1, further comprising a pharmaceutical carried by said hydrogel, and configured to be released upon degradation of the polymerized and cross-linked material.

7. A macromer solution for administration to the eye comprising:
a biocompatible pre-polymer material that is polymerized upon application of a specific biocompatible polymerization trigger wherein the pre-polymer material is configured to be selectively degradable after polymerization upon application of a degradation trigger.

8. The macromer solution of claim 7, wherein the solution produces eyesight enhancing properties upon polymerization.

9. The macromer solution of claim 7, wherein the pre-polymer material comprises a photo-initiator that begins polymerization of the pre-polymer material upon exposure to light.

10. The macromer solution of claim 7, wherein the pre-polymer material comprises a thermal polymerization initiator that begins polymerization of the pre-polymer material upon exposure to physiological temperatures.

11. The macromer solution of claim 7, wherein the pre-polymer material comprises a reactive polymerization agent that begins polymerization of the pre-polymer material upon exposure to a biocompatible reagent.

12. A method of treating the eye of a patient comprising the steps of:
applying the macromer of claim 7;
initiating polymerization of the macromer by applying a polymerization trigger; and
subsequently applying a degradation trigger to remove the polymerized macromer.

13. A method of adhering tissue comprising:
applying the macromer of claim 7 to the area to be adhered;
initiating a polymerization of the macromer by applying the polymerization trigger; and
subsequently applying the degradation trigger to remove the polymerized macromer.

14. A method of delivering a pharmaceutical treatment to a patient comprising:
applying the biocompatible hydrogel of claim 6; and
applying a degradation trigger.

15. A method of treating impaired eyesight comprising:
applying the macromer of claim 8 to the eye;
initiating polymerization of the macromer by applying the polymerization trigger; and
subsequently applying the degradation trigger to remove the polymerized macromer.

16. A biocompatible hydrogel for application to the eye comprising a polymer which comprises:
a hydrophilic core;
a polymerized and cross-linked material chemically linked to the hydrophilic core wherein the polymerized and cross-linked material comprises reactive sites configured to react to a specific degradation trigger and wherein the polymerized and cross-linked material may be degraded upon application of a degradation trigger.

17. The biocompatible hydrogel of claim 16, wherein the polymerized and cross-linked material upon degradation forms a material that may be absorbed through tear ducts of the eye.

18. The biocompatible hydrogel of claim 16, wherein the hydrophilic core is also degradable upon application of a degradation trigger.

19. The biocompatible hydrogel of claim 16, wherein the hydrophilic core is also degradable upon application of a second degradation trigger.

20. The biocompatible hydrogel of claim 16, wherein the degradation trigger is selected from the group consisting of a solution of differing pH, a salt, a salt solution, a weak acid solution, and an oxidizing compound.

21. The biocompatible hydrogel of claim 16, further comprising a pharmaceutical designed to be released upon degradation of the polymerized and cross-linked material.
